(19) United States
(12) Patent Application Publication
(10) Pub. No.: US 2006/0115421 A1
(43) Pub. Date: Jun. 1, 2006

(54) Process for separation by selective adsorption on a solid containing a zeolite with a crystalline structure analogous to IM-12

(76) Inventors: Philibert Leflaive, Bures sur Yvette (FR); Anne-Claire Dubreuil, Lyon (FR); Philippe Caullet, Illzach (FR); Joel Patarin, Flaxlanden (FR); Jean-Louis Paillaud, Mulhouse (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/256,271

(22) Filed: Oct. 24, 2005

(30) Foreign Application Priority Data
Oct. 29, 2004 (FR) 04/11.629

(51) Int. Cl. C01B 39/04; C01B 33/36; C01F 7/00

(52) U.S. Cl. 423/702

(57) ABSTRACT

A process for adsorption separation uses a solid IM-12 type adsorbent to separate a molecular species from any feed.

### FIELD OF THE INVENTION

The invention relates to a process for adsorption separation using, as the adsorbent mass, a solid containing a zeolite with a particular structure, analogous to that of IM-12.

Adsorption separation currently constitutes the technology of choice when technologies based on liquid-vapour equilibrium such as distillation cannot separate different species of a mixture.

Adsorption separation is widely used to separate and purify gas and liquid in many fields, from the petroleum, petrochemicals and chemicals industries to environmental and pharmaceutical applications.

Typical industrial applications for adsorption separation are the production of industrial gas (oxygen, nitrogen, hydrogen), separation of hydrocarbons (linear and branched paraffins, xylenes, for example), air, water and effluent treatments to eliminate pollutants (sulphur-containing compounds, volatile organic compounds, etc), drying, separating chiral isomers, etc.

### PRIOR ART

Adsorption separation processes are well known in the prior art.

A summary of the characteristics of that type of process can, for example, be found in volume B3 of Ullmann's Encyclopedia (p9-37 to 9-50) or in volume 4 of the “Handbook of porous solids”, Wiley & Sons.

Of all of the processes for adsorption separation, we may cite the process known as simulated counter current (SCC) described, for example, in United States patent U.S. Pat. No. 2,985,589 and French patent FR-A-2 681 066, the process known as pressure swing adsorption (PSA) described, for example, in U.S. Pat. No. 6,641,664, FR-A-2 655 980, FR-A-2 837 722 or FR-A-2 774 386 and the process known as thermal swing adsorption (TSA) described, for example, in U.S. Pat. No. 6,432,171 and European patent EP-A-1 226 860.

The principle of a process for adsorption separation resides in selective adsorption of one or more constituents on a microporous solid.

The adsorption solids may be of a number of types, for example zeolites or molecular sieves, silica gels, aluminas, activated charcoal.

All of those solids are characterized by a large specific surface area, of the order of 300 to 1200 m²/g. The zeolites are differentiated from other types of solid adsorbents in that they are microporous crystalline solids and adsorption takes place within the crystals. The term “microporous” means a pore size of less than 20 Å.

A large number of natural or synthetic zeolites exist and are recorded in the “Atlas of Zeolite Structure Types” by Ch Baerlocher, W M Meier and D H Olson, 5^{th} edition, review, 2001, Elsevier, published by the International Zeolite Association (IZA).

They are distinguished by their composition and crystalline structure.

The crystalline structure describes a two-dimensional or three-dimensional network of channels and/or pores of a defined size which constitutes the microporous volume.

The size of the openings which control access to said pores is also an important parameter in adsorption separation.

Of the zeolites which have been synthesized over about the past forty years, some solids have resulted in significant advances in the adsorption fields. These include Y zeolite (U.S. Pat. No. 3,130,007) and ZSM-5 zeolite (U.S. Pat. No. 3,702,886).

Of recently synthesized zeolites, IM-12, which is described in the Applicant's patent application 03/11333, may be mentioned. In addition to a novel crystalline structure, solid crystalline IM-12 has a chemical composition, expressed as the anhydrous base, in terms of moles of oxides, defined by the following general formula: XO₂:mYO₂:pZ₂O₃:qR_{2/n}O, in which R represents one or more cations with valency n, X represents one or more tetravalent elements other than germanium, Y represents germanium, and Z represents at least one trivalent element.

The letters m, p, q respectively represent the number of moles of YO₂, Z₂O₃ and R_{2/n}O, m being in the range 0 to 1, p being in the range 0 to 0.5 and q being in the range 0 to 0.7.

Said crystalline solid IM-12 has a novel topology with a two-dimensional system of interconnected channels comprising two types of straight channels defined by openings with 14 and 12 X and/or Y and/or Z atoms respectively, said atoms being 4-coordinate, i.e. surrounded by four oxygen atoms.

The term “pore diameter” is used as a functional definition of the size of a pore in terms of the size of molecule which can enter that pore. It does not define the actual dimension of the pore as it is usually difficult to determine since it is often irregular in shape (i.e. usually non-circular).

D W Breck provides a discussion of the effective pore diameter in his book entitled “Zeolite Molecular Sieves” (John Wiley & Sons, New York, 1974) on pages 633 to 641.

Since the cross sections of zeolite channels are rings of oxygen atoms, the pore size in zeolites may also be defined by the number of oxygen atoms forming the annular section of the rings, designated by the term “member rings”, MR.

As an example, the “Atlas of Zeolite Structure Types” by Ch Baerlocher, W M Meier and D H Olson, 5^{th} edition, review, 2001, Elsevier, indicates that zeolites with structure type FAU have a network of 12 MR crystalline channels, i.e. with a section constituted by 12 oxygen atoms. The crystalline solid IM-12 has a two-dimensional network of interconnected channels comprising two types of straight channels defined by 14 and 12 MR openings. This definition is well known to the skilled person and will be used below.

Adsorption separation is based on selective adsorption (either thermodynamic or kinetic) of the various gaseous or liquid constituents constituting the feed due to specific interactions between the surface of the adsorbent solid and the adsorbed molecules.

For simplification, we shall henceforth use the term “adsorbent” to designate the solid adsorbent and “adsorbate molecule” or “adsorbate” to designate the adsorbed species.

Adsorption separations may be based on steric, kinetic or thermodynamic equilibrium effects.

When a steric effect is involved, only molecules with a critical diameter less than the pore diameter are adsorbed in the adsorbent.

The various species contained in the mixture are thus separated as a function of the molecular size of those species.

A typical example of that type of separation is the separation of linear and branched alkanes using 5A zeolite as illustrated in the Applicant's patents EP-A-0 820 972 and U.S. Pat. No. 6,353,144.

In addition to the steric effect, the mixtures of molecular species may be separated by a kinetic effect if one of the species is adsorbed much faster or more slowly than the other species contained in the mixture.

Whether the steric or the kinetic effect dominates depends on the size and distribution of the micropores.

If the critical diameter of a molecule of adsorbate is comparable with that of the pores of the adsorbent, a steric and kinetic effect may be produced as the smallest adsorbate molecules may adsorb more rapidly. Such an effect occurs, for example, when separating multibranched paraffins on a zeolitic adsorbent with a mixed structure with principal channels having a 10 MR opening and secondary channels having an opening with at least 12 MR, as illustrated in the Applicant's patent FR-A-2 813 310.

That patent describes a process for separating multibranched paraffins from a hydrocarbon feed containing hydrocarbons containing 5 to 8 carbon atoms per molecule, in particular linear, monobranched and multibranched paraffins, using a zeolite with structure type NES (for example NU-85 or NU-86 zeolite).

Adsorption separations based on thermodynamic equilibrium effects are based on preferential adsorption of one of the compounds with respect to other compounds contained in the mixture to be separated. In the case of said separations termed “thermodynamic” separations, the adsorbent has a pore diameter that is larger than the critical diameter of the molecules to be separated, in fact as large as possible, to facilitate macroporous diffusion of molecules. One example of that type of separation is the separation of para-xylene from a feed containing xylenes and ethylbenzene on faujasite type zeolites the prior art of which is illustrated in the Applicant's patent EP-A-0 531 191.

One essential characteristic of adsorption technology is its transitory and generally cyclic function since, after an adsorption phase, the adsorbent solid must be partially or completely regenerated for subsequent use, i.e. it must be freed of adsorbed species, generally using a desorption solvent or by reducing the pressure (PSA processes) or by a temperature effect (TSA processes).

This dynamic function results in a certain complexity of adsorption processes as regards equipment, process control, dimensions and optimization of the adsorption and desorption cycles.

Separation performances depend not only on thermodynamic properties, but also on kinetic and hydrodynamic properties. In particular, the adsorbent should have as large a pore volume as possible and a pre size that is suitable for the desired separation type.

### BRIEF DESCRIPTION OF THE FIGURES

FIG. 1 shows the nitrogen adsorption isotherm at 77 K of the silicogermanate IM-12 synthesized using the method described in Example 1.

FIG. 2 shows a chromatographic representation of the separation of para-xylene from a para-xylene/ortho-xylene mixture at 150° C., with the same IM-12 silicogermanate and a desorbant constituted by pure toluene.

### BRIEF DESCRIPTION OF THE INVENTION

The invention concerns a group of processes for adsorption separation using an adsorbent characterized in that it contains a solid with a crystalline structure analogous to that of solid IM-12 and having a chemical composition, expressed as the anhydrous base in terms of moles of oxide, by the formula XO₂:mYO₂:pZ₂O₃:qR_{2/n}O, in which R represents one or more cations with valency n, X represents one or more tetravalent elements other than germanium, Y represents germanium and Z represents at least one trivalent element.

The mixture containing the molecular species to be separated may be any mixture of hydrocarbons, meaning that each species forming the mixture may contain any number of carbon atoms.

The molecular species to be separated from the hydrocarbon mixture does not have to be a hydrocarbon.

The invention is applicable in many and varied fields, from the petroleum, petrochemicals and chemical industries to environmental and pharmaceutical applications.

The process of the invention may be carried out in both the liquid and in the gas phases. The operating conditions for the separation unit depend on the yield and degree of purity of each of the desired streams. As an example, a cyclic PSA or TSA type process functions at temperatures and pressures which allow adsorption and desorption of the desired species. In general, the temperature is fixed at between about 0° C. and 400° C., and preferably between 50° C. and 250° C.

The pressure may be between about 0.01 MPa and about 15 MPa, preferably between about 0.05 MPa and about 5 MPa. Desorption is carried out in a number of manners, for example by reducing the pressure (PSA) or by increasing the temperature (TSA processes).

In the same manner, a simulated counter current process functions at a temperature which is usually fixed at between about 20° C. and 250° C., preferably between 60° C. and 210° C. The pressure is higher than the bubble pressure of the species to be separated, to maintain a liquid phase throughout the system. The volume ratio of the desorbant to the feed is generally in the range 0.5 to 30.

If one of the molecular species is an impurity, i.e. typically in a concentration of less than 1% by weight, and more particularly in the range 0.1% by weight to a few ppm by weight, the process of the invention may be reduced to passing the stream to be treated through one or more beds of adsorbents in a temperature range in the range −50° C. to 300° C., preferably in the range −50° C. to 100° C. The bed or beds may be regenerated using a purge gas which traverses the bed or beds in a temperature in the range from −50° C. to 300° C., preferably in the range −50° C. to 150° C. to desorb the impurity from the adsorbent.

The adsorbent will be adapted to the envisaged application. Thus, several parameters such as the ratio X/Ge, the ratio (X+Ge)/Z, the nature of the cation(s) R, will be adjusted to ensure optimal performance of the process. In the same manner, the form in which the adsorbent will be used (extrudates, powder, beads) will depend on the type of process used.

### DETAILED DESCRIPTION OF THE INVENTION

The invention provides a group of adsorption separation processes which will generically be termed a separation process, using an adsorbent characterized in that it contains a solid with a crystalline structure analogous to that of solid IM-12 and has a chemical composition, expressed as the anhydrous base in terms of moles of oxides, by the formula XO₂:mYO₂:pZ₂O₃:qR_{2/n}O, in which R represents one or more cations with valency n, X represents one or more tetravalent elements other than germanium, Y represents germanium, and Z represents at least one trivalent element. French patent application no 03/11333 describes the zeolite IM-12 and its separation process.

Compared with the prior art, the process of the invention has the following advantages:
When the desired effect is a steric and/or kinetic effect, the use of a zeolite with large pores allows large molecules to be separated. This has been envisaged with other zeolites such as CIT-5 (U.S. Pat. No. 6,043,179), SSZ-53 or SSZ-59 (Burton A et al, Chemistry: A Eur Journal, submitted), OSB-1, UTD-1F (Wessels T, Baerlocher C, McCusker L B, Creyghton E J, J Am Chem Soc 121, 6242-6247 (1999)), or AIPO-8 (Dessau R M, Schlenker J L, Higgins J B, Zeolites, 10, 522-24 (1990)), but the latter have a pore volume which is much smaller than that of IM-12. Further, IM-12 is the only one of said zeolites to have a two-dimensional system of channels the smallest diameter channel of which is more than 8 MR.
In the case in which separation is based on thermodynamic equilibrium, the process of the invention has the advantage of providing good separation quality, solid IM-12 having both a large capacity and wide straight channels defined by 14 and 12 MR openings, forming a two-dimensional system of interconnected channels. This two-dimensional system of large interconnected channels can in fact result in good diffusion of molecular species in the pores, thus reducing diffusional resistances of adsorbed molecular species.
Finally, in all cases, the solid IM-12 has high thermal stability, which is vital in order to avoid degradation of the solid employed, particularly in TSA type processes. In fact, the solid IM-12 may resist several calcining cycles at 600° C., which temperature is substantially higher than those generally encountered in adsorption separation processes (typically 400° C. maximum).

The crystalline structure of the crystalline solid IM-12 is a three-dimensional structure formed by tetrahedra. In particular, it comprises units of the double ring to four tetrahedral type. The peak of each tetrahedron is occupied by an oxygen atom. Solid crystalline IM-12 has a novel topology with a two-dimensional system of interconnected channels comprising two types of straight channels defined by openings with 14 and 12 X and/or Y and/or Z atoms respectively, said atoms being 4-coordinate, i.e. surrounded by four oxygen atoms.

The dimensions of said channels are respectively 9.5×7.1 Å for 14 MR channels and 8.5×5.1 Å for 12 MR channels.

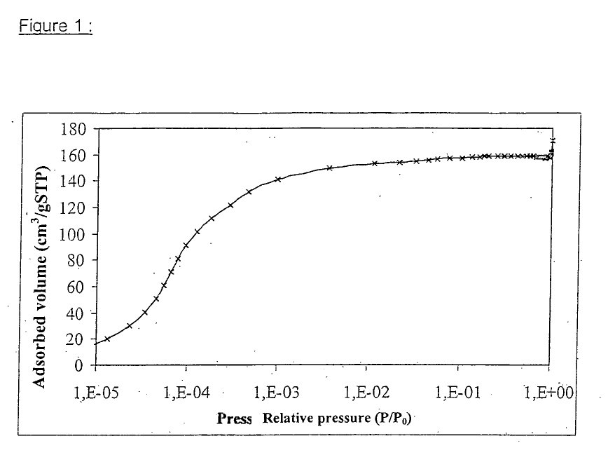

The nitrogen adsorption isotherm at 77 K of the silicogermanate IM-12 shown in FIG. 1 is characteristic of a purely microporous type “Ia” material as per the IUPAC nomenclature, indicating the absence of secondary micropores and mesoporosity. The microporous volume is 0.26 cm³/g and the BET specific surface area is 670 m²/g.

By way of comparison, faujasite type zeolites, which have among the highest microporous volumes and the largest pore openings, have a microporous volume of approximately 0.35 cm³/g measured by nitrogen adsorption at 77 K, and window diameters of 7.4×7.4 Å (“Atlas of Zeolite Structure Types” by Ch Baerlocher, W M Meier and D H Olson, 5^{th} edition, review, 2001, Elsevier, cited above. It should be noted that in the case of faujasites, part of the pore volume (the sodalite cages) is not accessible to molecules other than water and nitrogen. Thus, for example, the pore volume which is accessible to a multibranched alkane such as 2,2,4-trimethylpentane is 0.27 cm³/g.

The group of adsorption separation process aimed at separating a product or a group of products from a feed containing them form the subject matter of the present invention.

Thus, the invention is applicable in many and varied fields, from the petroleum, petrochemical and chemical industries to environmental and pharmaceutical applications.

More particularly, envisaged applications are the production of industrial gas (oxygen, nitrogen, hydrogen), the separation of hydrocarbons and elimination of pollutants (sulphur-containing compounds, volatile organic compounds, etc).

Preferably, the separations which concern the present invention are:
Separating one xylene isomer (ortho-, meta- or para-xylene) or ethylbenzene from a hydrocarbon feed essentially comprising C8 aromatic hydrocarbons. In this case, the technology used is preferably a simulated moving bed. The preferred desorbant is generally toluene, however other desorbants such as para-diethylbenzene, paradifluorobenzene or diethylbenzene mixtures may also be suitable.
Preferably, the ratio of the desorbant to the feed is in the range 0.5 to 2.5, more preferably in the range 1 to 2.
   The temperature is generally in the range 20° C. to 250° C., preferably in the range 90° C. to 210° C. and more particularly in the range 160° C. to 200° C., at a pressure in the range from aromatic pressure to 20 bars (1 bar=0.1 MPa).
Separating linear paraffins from a mixture of hydrocarbons containing them. Depending on the length of the paraffins to be separated, said separation may be carried out in the gas phase (light compounds) or in the liquid phase (heavy compounds). In the case in which said separation is carried out in the gas phase, a PSA type process is preferably used.
   The pressure in the column during the adsorption phase is preferably in the range 0.2 to 3 MPa, and during the desorption phase it is in the range 0.05 to 0.5 MPa. The desorbant used may be an inert gas such as hydrogen or nitrogen, or a hydrocarbon, such as C3-C6 paraffins.
   When the separation is carried out in the liquid phase, a simulated moving bed type process is preferably used. In this case, the operating temperature of the unit is preferably in the range 100° C. to 250° C. The pressure in the unit is preferably in the range 0.2 to 2 MPa. The desorbant used is preferably a hydrocarbon, in particular a C3-C6 paraffin or a mixture of C3-C6 paraffins.
Separation of linear and monobranched paraffins from multibranched paraffins in a mixture containing them. Depending on the length of the paraffins to be separated, said separation may be carried out in the gas phase (light compounds) or in the liquid phase (heavy compounds). When said separation is carried out in the gas phase, a PSA type process is preferably used. The pressure in the column during the adsorption phase is preferably in the range 0.2 to 3 MPa, and during the desorption phase, it is in the range 0.05 to 0.5 MPa.
The desorbant used may be an inert gas, such as hydrogen or nitrogen, or a hydrocarbon such as C3-C6 paraffins. Hydrogen is a particularly suitable desorbant for said separation, as it can be recycled directly to the isomerization reactor with the desorbate (effluent from the desorption unit rich in normal and branched paraffins).
When said separation is carried out in the liquid phase, a simulated moving bed type process is preferably used. In this case, the operating temperature of the unit is preferably in the range 100° C. to 250° C. The pressure in the unit is preferably in the range 0.2 to 2 MPa. The desorbant employed is preferably a hydrocarbon, in particular a C3-C6 paraffin or a mixture of C3-C6 paraffins.
Separation of one or more isomers of dimethylnaphthalene (for example 2,6-dimethylnaphthalene) from a feed of hydrocarbons essentially comprising C12 aromatic hydrocarbons. In this case, the technology used is preferably a simulated moving bed.
The preferred desorbant is generally toluene, but other desorbants such as paradiethylbenzene, paradifluorobenzene or diethylbenzene mixtures may also be suitable. Preferably, the ratio of desorbant to feed is in the range 0.5 to 2.5, more preferably in the range 1 to 2 by volume. The temperature is generally in the range 20° C. to 300° C., preferably in the range 90° C. to 260° C., and more particularly in the range 160° C. to 250° C. and the pressure is in the range from atmospheric pressure to 2 MPa, preferably 0.2 to 2 MPa.
Separating one or more olefins from a hydrocarbon feed essentially comprising olefins or essentially paraffins and olefins (for example separation of 1,3-butadiene from a mixture of 1,3-butadiene, isobutane, n-butane, isobutane, cis- and trans-2-butenes, ethane/ethylene separation, propane/propylene separation or the separation of isoprene from a mixture of C5 olefins).
Separating one of the isomers of dichlorobenzene (ortho-, meta- or para-dichlorobenzene) from a feed essentially comprising dichlorobenzenes. In this case, the technology used is preferably a simulated moving bed. The preferred desorbant is generally toluene, but other desorbants such as a mixture of para-xylene, metaxylene or xylenes may also be suitable. The temperature is generally in the range 20° C. to 250° C., preferably in the range 90° C. to 210° C., and more particularly in the range 120° C. to 200° C. and the pressure is in the range from atmospheric pressure to 2 MPa, preferably in the range 0.2 to 2 MPa.
Separating heavy aromatic compounds (polynuclear aromatics—PNA) present in hydrocracking residues. In this case, the adsorbent is generally placed in a fixed bed. Preferably, several beds placed in parallel or in series are used. The temperature and pressure during the adsorption phase are preferably selected to maintain the hydrocarbons in the liquid phase. The temperature is generally in the range 20° C. to 350° C., more particularly in the range 50° C. to 250° C., at a pressure in the range from atmospheric pressure to 4 MPa, preferably in the range 0.2 to 4 MPa.
Purification of a stream of hydrocarbons containing sulphur-containing and/or nitrogen-containing impurities (for example desulphurization of a gas oil or a gasoline). Preferably, the stream is hydrotreated in advance to reduce the amount of sulphur-containing and/or nitrogen-containing compounds to less than 500 ppm, and ideally to less than 50 ppm. During the adsorption phase, the temperature is generally in the range 20° C. to 400° C., preferably in the range 100° C. to 280° C., and more particularly in the range 150° C. to 250° C., at a pressure in the range 0.3 to 3 MPa.
Purification of a natural gas containing mercaptans. In this case, the technology used is preferably TSA (temperature swing adsorption0. The purification step is preferably carried out at a pressure in the range 2 to 10 MPa, and at a temperature in the range −40° C. to 100° C. The mercaptan desorption step is preferably carried out at a pressure in the range 0.5 to 10 MPa and at a temperature in the range 0° C. to 150° C.

The adsorbent is adapted to the envisaged application. Thus, several parameters such as the ratio X/Ge, the ratio (X+Ge)/Z, the nature of the cation or cations R, are adjusted to ensure optimum performance of the process. In the same manner, the form in which the adsorbent is used (extrudates, powder, beads) will depend on the type of process employed.

### EXAMPLES

The invention will be better understood from the following examples which illustrate the invention without, however, limiting its scope.

Example 1 illustrates adsorption separation based on a steric and kinetic effect.

Example 2 concerns the separation of xylenes.

Example 3 illustrates a process for separating ortho-xylene from a mixture of xylenes and ethylbenzene.

### Example 1

The hydrocracking reaction produces undesirable heavy aromatic compounds (HPNA, heavy polynuclear aromatics) which clog equipment and reduce catalyst service life. Their formation increases with conversion and the mean molecular weight of the feed.

In general, the unconverted fraction has to be recycled at the outlet from the reactor. During the operation, heavy aromatic compounds accumulate in this recycle. Said accumulation results in even more clogging of the reactor. Only the heaviest compounds, however, generate such problems. It is thus important to remove them from the recycle stream using a separation process. IM-12, with its very large pores, is an adsorbent of choice for said separation.

A IM-12 silicogermanate was produced in accordance with Example 1 of the Applicant's patent application no 03/11333. It consists of mixing, in a beaker:
5.78 g of an aqueous 20% solution of (6R,10S)-6,10-dimethyl-5-azoniaspiro[4,5]decane hydroxide (ROH); and
0.872 g of amorphous germanium oxide (Aldrich);
then, after dissolving the oxide with stirring, adding 2.5 g of colloidal silica (Ludox HS-40 (Aldrich)) and 6.626 g of water.

After homogenizing, the gel obtained was placed in an autoclave and heated for 6 days at 170° C., with stirring. After filtering, the product was washed with distilled water and dried at 70° C. The sample was then calcined in a muffle furnace in a constant stream of air at a maximum temperature of 550° C.

The silicogermanate IM-12 was obtained in its calcined form, and had the formula SiO₂: 0.23 GeO₂.

Table 1 below shows the kinetic diameters of various molecules containing one or more aromatic rings as calculated by Henry W Haynes Jr, Jon F Parcher and Norman E Heimer (Ind Eng Chem Process Des Dev, 22, 401-409 (1983)).

TABLE 1

| Molecules | Number of aromatic nuclei | Critical diameters (Å) |
|---|---|---|
| Benzene | 1 | 6.7 |
| Naphthalene | 2 | 7.3 |
| Anthracene | 3 | 7.3 |
| Phenanthrene | 3 | 7.8 |
| Pyrene | 4 | 9.0 |
| Coronene | 6 | 11.4 |

The dimensions of the IM-12 channels were respectively 9.5×7.1 Å for 14 MR channels and 8.5×5.1 Å for 12 MR channels. Adsorption separation based on a steric and kinetic effect can thus isolate products with a molecular weight that is greater than or less than that of coronene, such as ovalene (8 aromatic rings), their alkylated derivatives, dimers of coronene, and more generally any molecule with a molecular diameter greater than that of coronene.

For said separation, the adsorbent was placed in several fixed beds disposed in parallel. The temperature during the adsorption phase was in the range 50° C. to 250° C. and the pressure was in the range from atmospheric pressure to 4 MPa.

### Example 2

For this example, we carried out a drilling test (test 1) (frontal chromatography) to determine the ability of IM-12 to separate ortho-xylene from other xylenes.

IM-12 was synthesized using the method described Example 1.

The adsorbent was then placed in a column. The quantity used for each test was 2.63 g. For each test, the temperature of the column was kept at 150° C. and the pressure was sufficient to ensure that the phase was liquid, i.e. about 1 MPa. The desorbant used was toluene.

The effluent from the column was sampled (30 samples) then analyzed by gas chromatography to determine the composition of the effluent at various time intervals.

In a first test, the composition of the feed was as follows:

Para-xylene: 45% by weight;

Meta-xylene: 45% by weight;

Iso-octane: 10% by weight (used as a tracer to estimate non-selective volumes and not involved in separation).

In a second test (test 2), the composition of the feed was as follows:

Para-xylene: 50% by weight

Ortho-xylene: 50% by weight

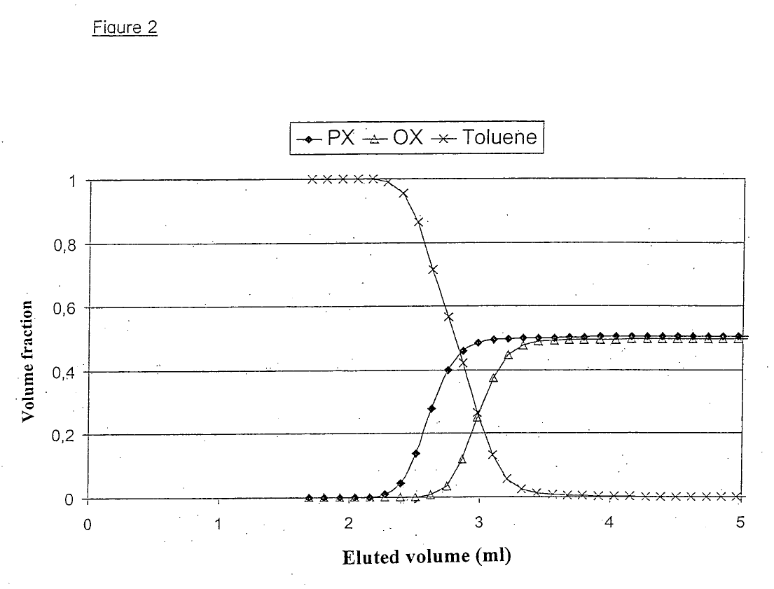

The drilling curve obtained corresponding to said feed is shown in FIG. 2.

The following operating mode was employed:
filling the column with sieve and placing in a test bench;
filling with solvent at ambient temperature;
progressive rise to 150° C. in stream of toluene (0.2 cm³/min);
solvent/feed permutation to inject feed (0.2 cm³/min);
feed injection then maintained for a period sufficient to reach thermodynamic equilibrium;
collect and analyze effluent.

The capacity of the sieve and its selectivity were then calculated and are shown in the following table. The selectivity α_{ox/px} was calculated from test 2. Test 1 allowed the selectivity α_{px/px} to be calculated. The selectivity α_{ox/px} was calculated as the product of the two preceding selectivities.

| | Capacity (g of C8 | Selectivity | | |
|---|---|---|---|---|
| Nature of solid | ads/g of sieve) | α_{ox/px} | α_{ox/mx} | Reference |
| IM-12 | 0.136 | 2.05 | 2.11 | In accordance |
| | | | | with invention |
| CSZ-1 (K⁺ form) | 0.12 | 1.4 | 2.4 | US-A-4 376 226 |
| CSZ-1 (Pb²⁺ | 0.08 | 2.1 | 2.8 | US-A-4 376 226 |
| form) | | | | |
| AlPO₄-5 | 0.057 | 2.6 | 2.7 | US-A-4 482 776 |
| NaX | * | 1.8 | 1.4 | US-A-4 482 777 |
| CaY | * | 2.4 | 1.8 | US-A-4 482 777 |
| AgX | * | 1.81 | 1.64 | US-A-4 529 828 |

* In contrast to frontal chromatography (drilling curves), the pulse experiments carried out here did not allow the capacity of the sieve to be calculated.

Compared with other adsorbents, it can be seen that IM-12 could produce satisfactory results for ortho-xylene separation.

The zeolite with the closest performance was CSZ-1 zeolite exchanged with lead. Clearly, the presence of heavy metals such as lead should be avoided for environmental reasons. Further, in all cases, the IM-12 had a larger pore size than the other adsorbents, which allowed better diffusion of molecules into the pores and thus a reduced matter transfer resistance.

### Example 3

Ortho-xylene was produced from a feed comprising a mixture of xylenes and ethylbenzene with the following composition by weight:

Para-xylene: 1.0% by weight

Meta-xylene: 63.8% by weight

Ortho-xylene: 28.0% by weight

Ethylbenzene: 7.2% by weight

using a simulated moving bed, in counter-current mode, the unit being composed of 24 equivalent beds, each bed having a volume of 381 cm³ and containing IM-12 produced using the method described in Example 1 and formed into beads. The solvent used was toluene.

The operating temperature was 150° C., the pressure at the recycle pump intake was kept at 1 MPa. All of the injected or withdrawn streams were under controlled flow rate, with the exception of the raffinate which was under pressure control.

There were 5 beds between the desorbant injection and the extract withdrawal, 9 beds between the extract withdrawal and the feed injection, 7 beds between the feed injection and the raffinate withdrawal, and 3 beds between the raffinate withdrawal and the desorbant injection. The following injection and withdrawal rates were used:

Feed: 25.2 cm³/min

Solvent: 37.8 cm³/min of toluene

Extract: 12.0 cm³/min

Raffinate: 51.0 cm³/min

Recycle flow rate (in zone 1): 134 cm³/min.

The valve permutation time (period) was 140 seconds.

The extract had the following composition:

Para-xylene: 0.01% by weight

Meta-xylene: 0.24% by weight

Ortho-xylene: 55.76% by weight

Ethylbenzene: 0.03% by weight

Toluene: 43.96% by weight

The raffinate had the following composition:

Para-xylene: 0.49% by weight

Meta-xylene: 31.47% by weight

Ortho-xylene: 0.72% by weight

Ethylbenzene: 3.55% by weight

Toluene: 63.77% by weight

After distilling the toluene, the extract obtained delivered 99.5% pure ortho-xylene in a yield of 94.8%.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure of all applications, patents and publications, cited herein and of corresponding French application No. 04/11.629, filed Oct. 29, 2004, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for adsorption separation of a molecular species from a mixture containing said species and other molecular species in any proportion, comprising bringing the mixture into contact with a solid adsorbent, the adsorbent being characterized in that it contains a solid with a crystalline structure analogous to that of IM-12 and having a chemical composition expressed, as the anhydrous base and in terms of moles of oxide, by the formula: XO₂:mYO₂:pZ₂O₃:qR_{2/n}O, in which R represents one or more cations with valency n, X represents one or more tetravalent elements other than germanium, Y represents germanium, and Z represents at least one trivalent element.

2. An adsorption separation process according to claim 1, in which the mixture containing the molecular species to be separated is a mixture of hydrocarbons.

3. An adsorption separation process according to claim 1, in which the molecular species to be separated from a mixture of hydrocarbons is not a hydrocarbon.

4. An adsorption separation process according to claim 1, characterized in that desorption of the adsorbate is carried out by reducing the pressure compared with the pressure used for adsorption.

5. An adsorption separation process according to claim 1, characterized in that desorption of the adsorbate is carried out by increasing the temperature with respect to the temperature used for adsorption.

6. An adsorption separation process according to claim 1, characterized in that the process is of the simulated counter current type.

7. An adsorption separation process according to claim 1, comprising separating a xylene isomer (ortho-, meta- or para-xylene) or ethylbenzene from a hydrocarbon feed essentially comprising C8 aromatic hydrocarbons.

8. An adsorption separation process according to claim 7 comprising separating of a xylene isomer (ortho-, meta- or para-xylene) or ethylbenzene from a hydrocarbon feed essentially comprising C8 aromatic hydrocarbons, the desorbant employed being toluene, the volume ratio of the desorbant to the feed being in the range 0.5 to 2.5, preferably in the range 1 to 2, the temperature being in the range 20° C. to 250° C., preferably in the range 90° C. to 210° C., and more particularly in the range 160° C. to 200° C., and the pressure being in the range from atmospheric pressure to 2 MPa.

9. An adsorption separation process according to claim 1 comprising separating of linear paraffins from any mixture of hydrocarbons containing them, said separation being carried out in the gas phase, using a PSA type process, the pressure in the column during the adsorption phase preferably being in the range 0.2 to 3 MPa and during the desorption phase in the range 0.05 to 0.5 MPa, the desorbant used being an inert gas such as hydrogen or nitrogen, or a hydrocarbon such as C3-C6 paraffins.

10. An adsorption separation process according to claim 1 comprising separating linear paraffins from any mixture of hydrocarbons containing them, said separation being carried out in the liquid phase, using a simulated moving bed type process, the temperature being in the range 100° C. to 250° C., and the pressure being in the range 0.2 to 2 MPa, the desorbant used preferably being a hydrocarbon, in particular a C3-C6 paraffin or a mixture of C3-C6 paraffins.

11. An adsorption separation process according to claim 1 comprising separating linear paraffins and monobranched paraffins from multibranched paraffins in a mixture containing them, said separation being carried out in the gas phase by a PSA type process, the pressure in the column during the adsorption phase being in the range 0.2 to 3 MPa and during the desorption phase in the range 0.05 to 0.5 MPa, the desorbant used being an inert gas such as hydrogen or nitrogen or a hydrocarbon such as C3-C6 paraffins.

12. An adsorption separation process according to claim 1 comprising separating linear paraffins and monobranched paraffins from multibranched paraffins in a mixture containing them, said separation being carried out by means of a simulated moving bed process.

13. An adsorption separation process according to claim 1 comprising separating one or more isomers of dimethylnaphthalene from a hydrocarbon feed essentially comprising aromatic C12 hydrocarbons, said separation being carried out in a simulated moving bed, the preferred desorbant being toluene, the volume ratio of the desorbant to the feed being in the range 0.5 to 2.5, preferably in the range 1 to 2, the temperature generally being in the range 20° C. to 300° C., preferably in the range 90° C. to 260° C., and more particularly in the range 160° C. to 250° C., and the pressure being in the range from atmospheric pressure to 2 MPa.

14. An adsorption separation process according to claim 1 comprising separating one or more olefins from a hydrocarbon feed essentially comprising olefins or essentially comprising paraffins and olefins.

15. An adsorption separation process according to claim 1 comprising separating one or more isomers of dichlorobenzene (ortho-, meta- or para-dichlorobenzene) from a feed essentially comprising dichlorobenzenes, said separation being carried out in a simulated moving bed, the preferred desorbant being toluene, para-xylene, meta-xylene or a mixture of xylenes, the temperature being in the range 20° C. to 250° C., preferably in the range 90° C. to 210° C., and more particularly in the range 120° C. to 200° C., and the pressure being in the range from atmospheric pressure to 2 MPa.

16. An adsorption separation process according to claim 1 comprising separating heavy aromatic compounds (polynuclear aromatics—PNA) present in hydrocracking residues, the adsorbent being used in a fixed bed, the temperature generally being in the range 20° C. to 350° C., more particularly in the range 50° C. to 250° C., and the pressure being in the range from atmospheric pressure to 4 MPa.

17. An adsorption separation process according to claim 1 comprising purifying a stream of hydrocarbons containing sulphur-containing and/or nitrogen-containing impurities, the amount of sulphur-containing and/or nitrogen-containing compounds being less than 500 ppm, preferably less than 50 ppm, the temperature during the adsorption phase being in the range 20° C. to 400° C., preferably in the range 100° C. to 280° C., more particularly in the range 150° C. to 250° C., and the pressure being in the range 0.3 to 15 MPa.

18. An adsorption separation process according to claim 1 comprising purifying a natural gas containing mercaptans, said separation being carried out using TSA technology, the adsorption phase being carried out at a pressure in the range 2 MPa to 10 MPa, and at a temperature in the range −40° C. to 100° C., and the mercaptan desorption phase preferably being carried out at a pressure in the range 0.5 to 10 MPa, and at a temperature in the range 0° C. to 150° C.
